(19) United States
(12) Patent Application Publication
(10) Pub. No.: US 2019/0021667 A1
(43) Pub. Date: Jan. 24, 2019

(54) METHOD AND DEVICE FOR MONITORING BODY DATA BASED ON UNDERWEAR

(71) Applicant: TAI HING PLASTIC METAL LTD., Hong Kong (CN)

(72) Inventor: Chi Wing LIN, Hong Kong (CN)

(21) Appl. No.: 15/755,697

(22) PCT Filed: Oct. 19, 2017
(86) PCT No.: PCT/IB2017/056489

§ 371 Date: Feb. 27, 2018

(30) Foreign Application Priority Data
Oct. 19, 2016 (HK) 16112045.2

(51) Int. Cl. A61B 5/00; A61B 5/0205; A41B 9/00

(52) U.S. Cl. CPC A61B 5/6804; A61B 5/746; A61B 5/02055; A61B 5/0816; A41B 9/00; A61B 2562/0219; A61B 5/742

(57) ABSTRACT

The present invention relates to an underwear-based body data monitoring method and device. The method includes: receiving position data disposed on underwear and respiration data disposed on underwear; calculating displacement, velocity and acceleration; determining whether the displacement, velocity and acceleration all exceed a preset threshold; further determining whether the respiration data is within a preset respiration range when the displacement, velocity and acceleration all exceed a preset threshold; and outputting alarm information when the respiration data is not within a preset respiration range. By the underwear-based body data monitoring method and device, body data can be collected by sensors disposed on underwear in real time. In addition, it is able to roughly determine whether the physical condition is abnormal by the displacement, velocity and acceleration, and then further determine the physical condition by respiration data. The analysis process is simple and the analysis result is accurate and reliable.

### TECHNICAL FIELD

The present invention relates to the field of intelligent underwear and in particular to an underwear-based body data monitoring method and device.

### BACKGROUND

Underwear, as close-fitting clothing, includes vests, undershirts, undershorts, bras and the like. Underwear is clothing worn under other clothes, usually next to the skin. It is one of essentials in modern society.

At present, detection to the heart rate, mammary gland hyperplasia, and sleep quality of females is usually done by movable instruments or wearable wrist devices. However, the use of movable instruments can realize only simple measurements, the operation is relatively complicated, and it is unable to detect the body continuously. Although the use of wearable wrist devices can detect the body continuously, there is only a single standard to determine the physical condition and the analysis result is less reliable.

### SUMMARY

Accordingly, it is necessary to provide an underwear-based body data monitoring method and device, by which body data can be obtained in real time and the analysis result is accurate.

An underwear-based body data monitoring method is provided, comprising the following steps of:

receiving position data collected by a multi-axis sensor disposed on underwear and respiration data collected by a respiration sensor disposed on underwear;

calculating displacement, velocity and acceleration according to the position data;

determining whether the displacement, velocity and acceleration all exceed a preset threshold;

further determining whether the respiration data is within a preset respiration range when the displacement, velocity and acceleration all exceed a preset threshold; and

outputting alarm information when the respiration data is not within a preset respiration range.

In one embodiment, the method further comprises the following steps of:

receiving heartbeat data collected by a pulse sensor disposed on underwear and blood oxygen data collected by a blood oxygen sensor disposed on underwear; and

further determining whether the heartbeat data is within a preset heartbeat range and further determining whether the blood oxygen data is within a preset blood oxygen range, while performing the step of further determining whether the respiration data is within a preset respiration range; and

the step of outputting alarm information further comprises a step of:

outputting alarm information when the heartbeat range is not within a preset heartbeat range or when the blood oxygen data is not within a preset blood oxygen range.

In one embodiment, the method further comprises the following steps of:

receiving tension data collected by a tension sensor disposed on underwear;

determining whether the tension data is higher than a preset tension value;

further determining whether it is able to continuously receive body data collected by other sensors disposed on underwear, when the tension data is higher than a preset tension value; and

outputting alarm information when it is unable to continuously receive body data collected by other sensors disposed on underwear.

In one embodiment, the method further comprises the following steps of:

playing bedtime multimedia information when a preset bedtime arrives, and continuously receiving and storing body data collected by sensors disposed on underwear;

determining whether body data collected by sensors disposed on underwear is within a corresponding sleep data range;

stopping playing the bedtime multimedia information when body data collected by sensors disposed on underwear is within a corresponding sleep data range; and

playing wakeup multimedia information when a preset wakeup time arrives.

In one embodiment, the method further comprises the following steps of:

receiving temperature data collected by a temperature sensor disposed on underwear; and

potting and displaying a 3D Cartesian coordinate system with temperature data as an x-axis, heartbeat data as a z-axis and respiration data as a y-axis, or potting and displaying a 3D Cartesian coordinate system with temperature data as an x-axis, heartbeat data as a z-axis and blood oxygen data as a y-axis.

In one embodiment, the method further comprises the following steps of:

receiving heartbeat sound data and ambient sound data collected by a sound recording device disposed on underwear.

An underwear-based body data monitoring device is provided, comprising:

a data receiving module configured to receive position data collected by a multi-axis sensor disposed on underwear and respiration data collected by a respiration sensor disposed on underwear;

a data processing module configured to calculate displacement, velocity and acceleration according to the position data;

a data analysis module configured to determine whether the displacement, velocity and acceleration all exceed a preset threshold, and further determine whether the respiration data is within a preset respiration range when the displacement, velocity and acceleration all exceed a preset threshold; and

a data output module configured to output alarm information when the respiration data is not within a preset respiration range.

In one embodiment, the data receiving module is further configured to receive heartbeat data collected by a pulse sensor disposed on underwear and blood oxygen data collected by a blood oxygen sensor disposed on underwear;

the data analysis module is further configured to further determine whether the heartbeat data is within a preset heartbeat range and further determine whether the blood oxygen data is within a preset blood oxygen range, when the displacement, velocity and acceleration all exceed a preset threshold; and

the data output module is further configured to output alarm information when the heartbeat range is not within a preset heartbeat range or when the blood oxygen data is not within a preset blood oxygen range.

In one embodiment, the data receiving module is further configured to receive tension data collected by a tension sensor disposed on underwear;

the data analysis module is further configured to determine whether the tension data is higher than a preset tension value, and further determine whether it is able to continuously receive body data collected by other sensors disposed on underwear when the tension data is higher than a preset tension value; and

the data output module is further configured to output alarm information when it is unable to continuously receive body data collected by other sensors disposed on underwear.

In one embodiment, the device further comprises:

a time determination module configured to determine whether a preset bedtime arrives or whether a preset wakeup time arrives;

a multimedia playback module configured to play bedtime multimedia information when a preset bedtime arrives and stop playing the bedtime multimedia information when body data collected by sensors disposed on underwear is within a corresponding sleep data range, and play wakeup multimedia information when a preset wakeup time arrives;

a data storage module configured to store body data collected by sensors disposed on underwear; and

the data analysis module is further configured to determine whether body data collected by sensors disposed on underwear is within a corresponding sleep data range.

In one embodiment, the data receiving module is further configured to receive temperature data collected by a temperature sensor disposed on underwear; and

the device further comprises:

a display module configured to plot and display a 3D Cartesian coordinate system with temperature data as an x-axis, heartbeat data as a z-axis and respiration data as a y-axis, or plot and display a 3D Cartesian coordinate system with temperature data as an x-axis, heartbeat data as a z-axis and blood oxygen data as a y-axis.

In one embodiment, the data receiving module is further configured to receive heartbeat sound data and ambient sound data collected by a sound recording device disposed on underwear.

By the underwear-based body data monitoring method and device, body data can be collected by sensors disposed on underwear in real time. In addition, it is able to roughly determine whether the physical condition is abnormal by the displacement, velocity and acceleration, and then further determine the physical condition by respiration data. The analysis process is simple and the analysis result is accurate and reliable.

### BRIEF DESCRIPTION OF THE DRAWINGS

FIG. 1 is a flowchart of an underwear-based body data monitoring method in an embodiment;

FIG. 2 is a flowchart of an underwear-based body data monitoring method in another embodiment;

FIG. 3 is a flowchart of an underwear-based body data monitoring method in yet another embodiment;

FIG. 4 is a schematic diagram of data display in one embodiment;

FIG. 5 is a schematic diagram of an underwear-based body data monitoring device in an embodiment; and

FIG. 6 is a schematic diagram of an underwear-based body data monitoring device in another embodiment,

in which:
110 data receiving module
120 data processing module
130 data analysis module
140 data output module
150 time determination module
160 multimedia playback module
170 data storage module
180 display module

### DETAILED DESCRIPTION OF THE DISCLOSURE

To make the objectives, technical solutions and advantages of the present invention clearer, the present invention will be further described in detail as below with reference to the accompanying drawings by embodiments. It should be understood that specific embodiments described here are merely used for explaining but not limiting the present invention.

Before describing embodiments of the present invention in detail, it should be noted that the embodiments focus on combinations of method steps and system components related to the underwear-based body data monitoring method and device. Therefore, those method steps and system components have been marked by general symbols at appropriate positions in the drawings, and only details related to the understanding of embodiments of the present invention are shown to avoid details which are apparent to a person of ordinary skill in the art from obscuring the disclosure of the present invention.

Herein, relational terms, such as “left and right”, “upper and lower”, “before and after” and “first and second”, are merely used for distinguishing one entity or action from another entity or action without necessarily requiring or implying any actual such relationship or order between such entities or actions. Terms “comprising”, “including” or any other variants thereof are intended to cover non-exclusive inclusion, such that the processes, methods, articles or devices including a series of elements comprise not only the elements, but also other elements that are not explicitly listed or the inherent elements of the articles or devices.

In one embodiment, intelligent underwear is provided, on which various sensors and a storage chip are disposed. Those sensors can collect body data of a user who wears the intelligent underwear in real time. After such body data is collected, they may be saved in the storage chip or transmitted by wireless communication to a specified terminal or server, for example, the mobile phone, computer or tablet of the user or a server provided by the developer. A corresponding APP may be installed in the mobile phone, computer or tablet of the user. In addition, an interface may be reserved on the intelligent underwear, by which those body data can be transmitted by wired communication to a specified terminal or the like.

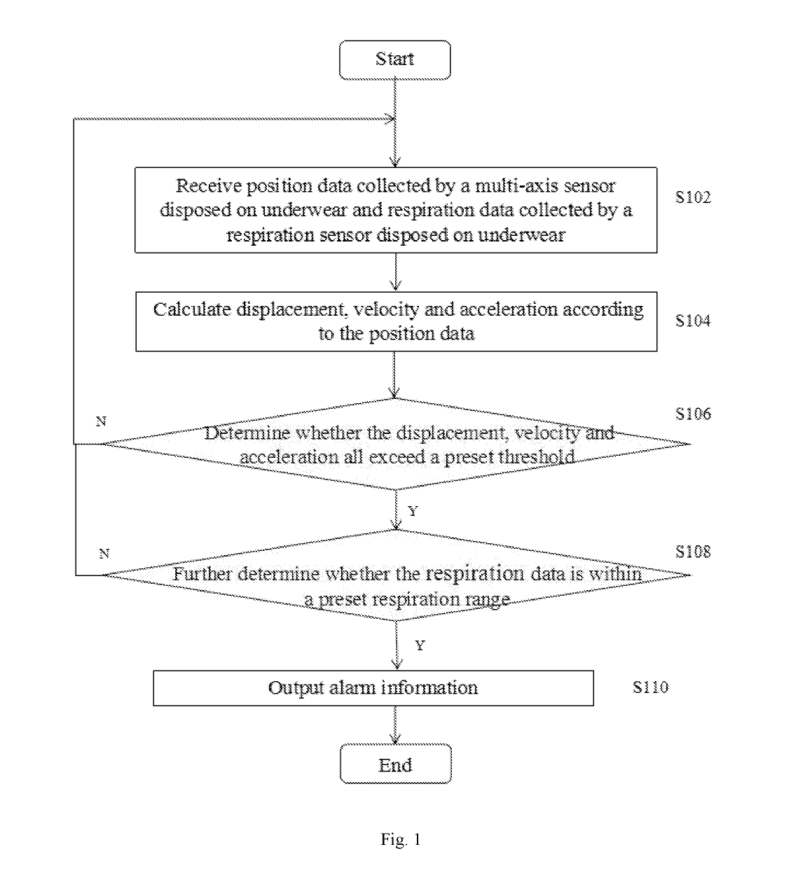

Referring to FIG. 1 which is a flowchart of an underwear-based body data monitoring method in on embodiment, the method may comprise the following steps.

S102: Position data collected by a multi-axis sensor disposed on underwear and respiration data collected by a respiration sensor disposed on underwear are received.

Specifically, the multi-axis sensor may be a three-axis sensor, a nine-axis sensor or the like. The three-axis sensor may be a gyroscope. The position data of the body may be collected by the multi-axis sensor. The position data includes, but is not limited to, three-axis acceleration, three-axis angular velocity, pitch angle, roll angle and yaw angle. The respiration sensor can collect chest tension of the body, and thus can detect the respiration rate, fluctuation intensity, frequency and the like. In other embodiments, other sensors may be disposed on the underwear. Data collected by those sensors may be sent to the mobile phone, tablet and computer of the user or to the server or the like, to be processed by the mobile phone, tablet and computer or by the server.

S104: Displacement, velocity and acceleration are calculated according to the position data.

Specifically, the displacement, velocity and acceleration of the user may be calculated according to the position data collected by the multi-axis sensor, for example, the three-axis acceleration, three-axis angular velocity, pitch angle, roll angle and yaw angle. The velocity may be obtained by multiplying the acceleration by a corresponding time, and the displacement may be obtained by multiplying the velocity by a corresponding time.

S106: It is determined whether the displacement, velocity and acceleration all exceed a preset threshold.

Specifically, body data may be collected in real time by sensors disposed on the intelligent underwear in this embodiment. By the body data, the body of the user may be monitored in real time. The corresponding body data will change when the user falls down or is attacked. For example, when the user falls down, the displacement, velocity and acceleration of the user may all exceed those in normal conditions. A preset threshold may be set in advance for each of the displacement, velocity and acceleration in standard conditions, wherein the preset threshold may be adjusted according to the monitored data when in use. That is, the user may use an average of data within a certain time span (for example, one hour, one day, one week, fifteen days, one month) as the threshold, or may define the threshold. In this way, when the calculated displacement, velocity and acceleration all exceed the respective preset threshold, it may be considered that the user falls down. Given that set(m) denotes a preset threshold for the displacement, set(m/s) denotes a preset threshold for the velocity, and set(m/s²) denotes a preset threshold for the acceleration, the above determination may be made by the following formulae:

$$\begin{matrix}{\sum }_{K = 0}^{n} ( X , Y , Z ) > set ( m ) & ( 1 ) \\ {\sum }_{k = 0}^{n} ( X \frac{m}{s} , Y \frac{m}{s} , Z \frac{m}{s} ) > set ( m / s ) & ( 2 ) \\ {\sum }_{k = 0}^{n} ( X \frac{m}{{s}^{2}} , Y \frac{m}{{s}^{2}} , Z \frac{m}{{s}^{2}} ) > set ( m / {s}^{2} & ( 3 )\end{matrix}$$

where, n and k are both positive integers, k=1, 2, 3 . . . n, representative of discrete time.(X,Y,Z) denotes the displacement of the user in the 3D coordinate system,

$$( X \frac{m}{s} , Y \frac{m}{s} , Z \frac{m}{s} )$$

denotes the velocity of the user in the 3D coordinate system, and

$$( X \frac{m}{{s}^{2}} , Y \frac{m}{{s}^{2}} , Z \frac{m}{{s}^{2}} )$$

denotes the acceleration of the user in the 3D coordinate system. When the displacement, velocity and acceleration all satisfy the above formulae, it may be roughly considered that the user falls down. In addition, a determination may also be made in the same way when the user is knocked down or fainted. The only difference is the respective threshold. A threshold may be set, in the same way, for the displacement, velocity and acceleration of the user in the cases where the user is knocked down or fainted, and will not be repeated here.

S108: It is further determined whether the respiration data is within a preset respiration range when the displacement, velocity and acceleration all exceed a preset threshold.

There are many situations satisfying the above condition in practical applications. For example, when the user wants to sleep, he/she may directly flop into bed. In this case, the body of the user is not under threat, but his/her displacement, velocity and acceleration may all exceed the preset threshold. If an alarm is given now, it is a false alarm. The use experience of the user is degraded. Therefore, in this embodiment, the current body state is determined also according to the respiration data collected by a respiration sensor. In this way, the accuracy of determination is further ensured.

S110: Alarm information is output when the respiration data is not within a preset respiration range.

In one embodiment, as a way of outputting alarm information, it is possible to make a shout out by the mobile phone, tablet, computer or other electronic devices of the user, or make a call to a family member in the contacts by the mobile phone, tablet and computer of the user, or directly make a call to the police. The specific way of outputting alarm information may be set by the user. For example, only one of the above three ways may be used, or several of those ways may be used. When several of those ways are used, the user may set priority for them. The user may further set family members in the contacts in advance. In this embodiment, the preset respiration range may be adjusted according to the monitored data when in use. That is, the user may use an average of data within a certain time span (for example, one hour, one day, one week, fifteen days, one month) as the preset respiration range, or may define the preset respiration range.

By the underwear-based body data monitoring method, body data can be collected by sensors disposed on underwear in real time. In addition, it is able to roughly determine whether the physical condition is abnormal by the displacement, velocity and acceleration, and then further determine the physical condition by respiration data. The analysis process is simple and the analysis result is accurate and reliable.

In one implementation, the method may further comprise a step of receiving heartbeat data collected by a pulse sensor disposed on underwear and blood oxygen data collected by a blood oxygen sensor disposed on underwear. In other words, a pulse sensor and a blood oxygen sensor may also be provided on the underwear. It may make a mistake in determining whether the body of the user is abnormal only by the respiration data collected by the respiration sensor. Therefore, in this embodiment, heartbeat data and blood oxygen data are additionally provided. It may be considered that the body of the user is abnormal once one of heartbeat data, blood oxygen data and respiration data becomes abnormal. In other embodiments, other standards may also be used, for example, temperature data collected by a temperature sensor, respiratory sound collected by a respiratory sound sensor, or the like. In one embodiment, it may be further determined whether the heartbeat data is within a preset heartbeat range and further determined whether the blood oxygen data is within a preset blood oxygen range, while performing the step of further determining whether the respiration data is within a preset respiration range. The step of outputting alarm information may further comprise a step of: outputting alarm information when the heartbeat range is not within a preset heartbeat range or when the blood oxygen data is not within a preset blood oxygen range. In this embodiment, the preset heartbeat range and the preset blood oxygen range may be adjusted according to the monitored data when in use. That is, the user may use an average of data within a certain time span (for example, one hour, one day, one week, fifteen days, one month) as the preset heartbeat range and the preset blood oxygen range, or may define the preset heartbeat range and the preset blood oxygen range.

In this embodiment, after it is roughly determined that the user may be under threat, it is accurately determined whether the user is under threat further by body data such as heartbeat data, blood oxygen data and respiration data. The way of determination is simple, and the result of determination is more accurate. Accordingly, the alarming function is more accurate. The range of application is wide.

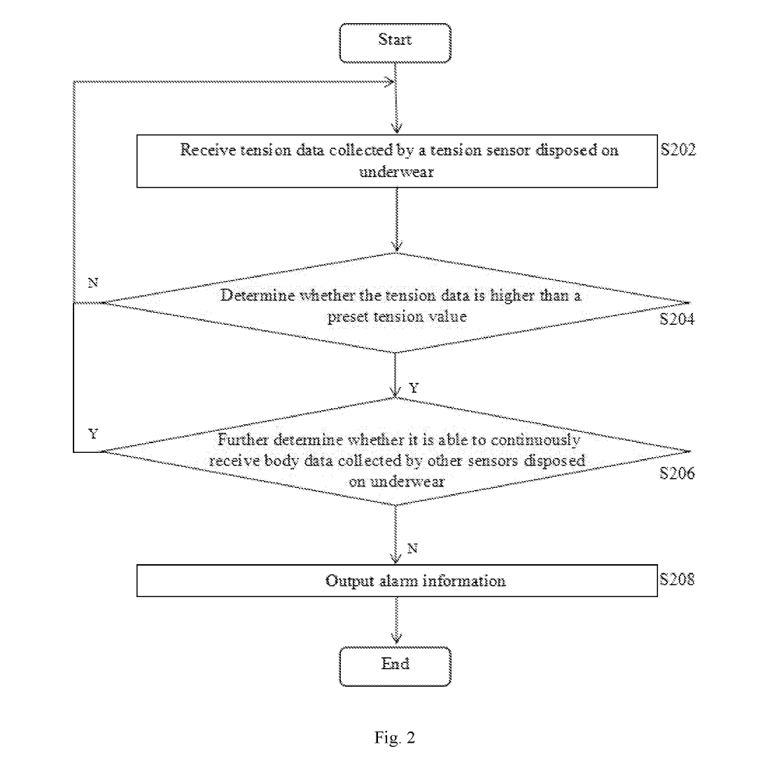

FIG. 2 is a flowchart of an underwear-based body data monitoring method in another embodiment. In this embodiment, the method may comprise the following steps.

S202: Tension data collected by a tension sensor disposed on underwear is received.

In on embodiment, a tension sensor may be provided on the underwear, for example, disposed between two cups of the bra. The tension may be sensed by the tension sensor, and further transmitted to a corresponding terminal, for example, the mobile phone, tablet or computer.

S204: It is determined whether the tension data is higher than a preset tension value.

Specifically, when the underwear is forcibly removed, the tension sensor may sense tension data that is higher than a preset tension value. Therefore, when the underwear of the user is forcibly removed, for example, when the user is raped, alarm information can be sent in time to ensure the safety of the user. In this embodiment, the preset tension value may be adjusted according to the monitored data when in use. That is, the user may use an average of data within a certain time span (for example, one hour, one day, one week, fifteen days, one month) as the preset tension value, or may define the preset tension value.

S206: It is further determined whether it is able to continuously receive body data collected by other sensors disposed on underwear, when the tension data is higher than a preset tension value.

Specifically, when in use of the underwear, the tension data collected by the tension sensor may also be higher than a preset tension value when the user adjusts the underwear, but the user is not raped. Therefore, to ensure the accuracy of determination, in this embodiment, it may be further determined whether it is unable to receive body data collected by other sensors disposed on the underwear after the tension data is higher than the preset tension value. For example, if the heartbeat data, the blood oxygen data and the like are lost after the tension data is higher than the preset tension value, it may be determined that the user is under threat.

S208: Alarm information is output when it is unable to continuously receive body data collected by other sensors disposed on the underwear.

In one embodiment, as a way of outputting alarm information, it is possible to make a shout out by the mobile phone, tablet, computer or other electronic devices of the user, or make a call to a family member in the contacts by the mobile phone, tablet and computer of the user, or directly make a call to the police. The specific way of outputting alarm information may be set by the user. For example, only one of the above three ways may be used, or several of those ways may be used. When several of those ways are used, the user may set priority for them. The user may further set family members in the contacts in advance.

By the underwear-based body data monitoring method, the tension data can be collected by the tension sensor disposed on underwear in real time, to roughly determine whether the user is under threat. Then, it is accurately determined whether the user is under threat further by whether it is able to continuously receive body data collected by other sensors disposed on the underwear. The analysis process is simple and the analysis result is accurate and reliable. The range of application is wide.

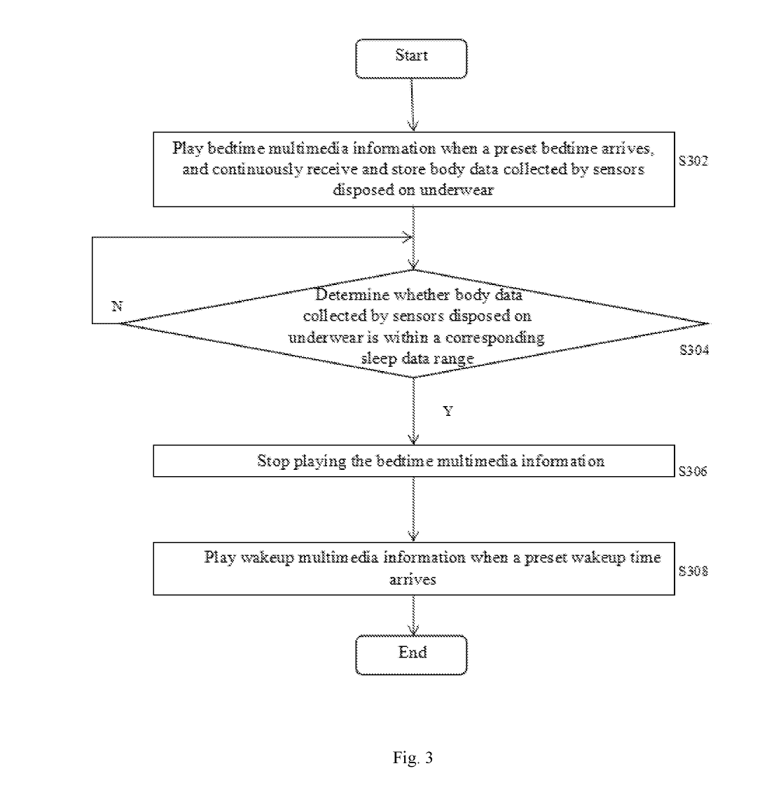

FIG. 3 is a flowchart of an underwear-based body data monitoring method in yet another embodiment. In this embodiment, the method may comprise the following steps.

S302: Bedtime multimedia information is played when a preset bedtime arrives, and body data collected by sensors disposed on underwear is continuously received and stored.

Specifically, the user may set a bedtime in advance, for example, 10 pm or 11 pm. Furthermore, the user may set a different bedtime in different days, for example, 10 pm from Sunday to Thursday and 11 pm in Friday and Saturday. After the user settles on the bed, bedtime multimedia information will be played. This can help the user to fall asleep. For example, soothing music may be played. The bedtime multimedia information may be set by the user as required. And, to monitor the sleep state of the user, body data of the user may be continuously collected after the user falls asleep and then stored, so that the user can view his/her sleep state. For example, the deep sleep time and light sleep time, body temperature during the sleep, heartbeat data and the like may be provided, and shown in broken-line graphs, histograms or the like.

S304: It is determined whether body data collected by sensors disposed on underwear is within a corresponding sleep data range.

In this embodiment, body data collected by the sensors may be synthetically judged to determine whether the user falls asleep. Or, several of such body data may be selected and individually judged. Or, several of such body data may be synthetically judged and then compared with a preset synthetic threshold. For example, in one embodiment, it may be determined whether the user falls asleep, by determining whether the body position data collected by the multi-axis sensor, the heartbeat data collected by the pulse sensor, and the blood oxygen data collected by the blood oxygen sensor are all within a corresponding sleep data range. In this embodiment, the sleep data range may be adjusted according to the monitored data when in use. That is, the user may use an average of data within a certain time span (for example, one hour, one day, one week, fifteen days, one month) as the sleep data range, or may define the sleep data range.

S306: The playing of the bedtime multimedia information is stopped when body data collected by sensors disposed on underwear is within a corresponding sleep data range.

Specifically, also in the above embodiment, if the body position data collected by the multi-axis sensor, the heartbeat data collected by the pulse sensor, and the blood oxygen data collected by the blood oxygen sensor are all within a corresponding sleep data range, it is considered that the user falls asleep. In this case, if the bedtime multimedia information is still being played, the bedtime multimedia information is regarded as noise which will influence the sleep of the user. Therefore, in one embodiment, the playing of the bedtime multimedia information may be stopped directly. In another embodiment, the bedtime multimedia information may be gradually decreased in volume and finally stopped.

S308: Wakeup multimedia information is played when a preset wakeup time arrives.

Specifically, the user may set a wakeup time in advance, for example, 7 am or 7:30 am or the like. Furthermore, the user may set a different bedtime in different days, for example, 7 am from Monday to Friday and 8 am in Saturday and Sunday. When the wakeup time arrives, if the user is still sleeping, wakeup multimedia information may be played to wake up the user. For example, joyful music may be played. Similarly, the wakeup multimedia information may be set by the user as required. After the user wakes up, he/she can stop the wakeup multimedia information manually. Or, the user may preset, in advance, a period of time after which the multimedia information will be automatically stopped, for example, 10 mins or 15 mins.

By the underwear-based body data monitoring method, the sleep state of the user can be monitored by continuously receiving and storing body data collected by the sensors disposed on the underwear. In addition, playing the bedtime multimedia information can help the user to fall asleep rapidly and playing the wakeup multimedia information can wake up the user.

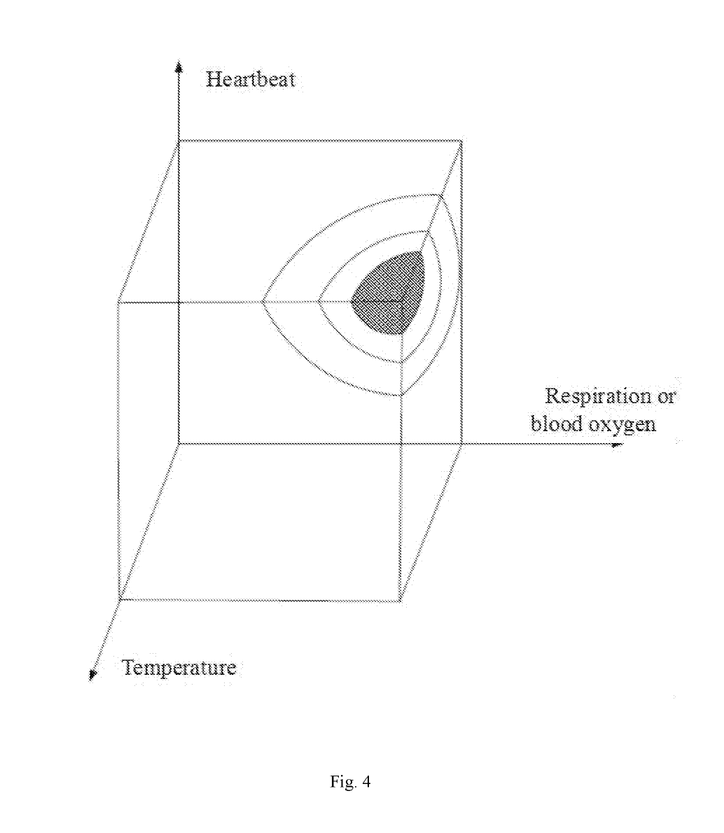

FIG. 4 is a schematic diagram of data display in one embodiment. In this embodiment, temperature data collected by a temperature sensor disposed on underwear may also be received. A corresponding graph may be plotted according to the received body data, and displayed to the user. For example, a 3D Cartesian coordinate system with temperature data as an x-axis, heartbeat data as a z-axis and respiration data as a y-axis may be plotted and displayed, or a 3D Cartesian coordinate system with temperature data as an x-axis, heartbeat data as a z-axis and blood oxygen data as a y-axis may be plotted and displayed. In other embodiments, other graphs may be plotted for the purpose of data display. In this way, in this embodiment, the body data of the user may be saved, and in some circumstances, may be processed and displayed to a corresponding person, for example, displayed to the user. In one embodiment, the corresponding display may be viewed by a doctor when the user goes to hospital.

In one embodiment, when the collected body data is not within a normal range, it may be highlighted in a color in the display, or filled with a color, to remind the user or the doctor.

In one embodiment, the method may further comprise a step of receiving heartbeat sound data and ambient sound data collected by a sound recording device disposed on underwear. The heartbeat sound data and ambient sound data may be shown to the doctor, or sent to a family member or to the police together with the alarm information, so that the doctor, the family member or the police can understand the situation.

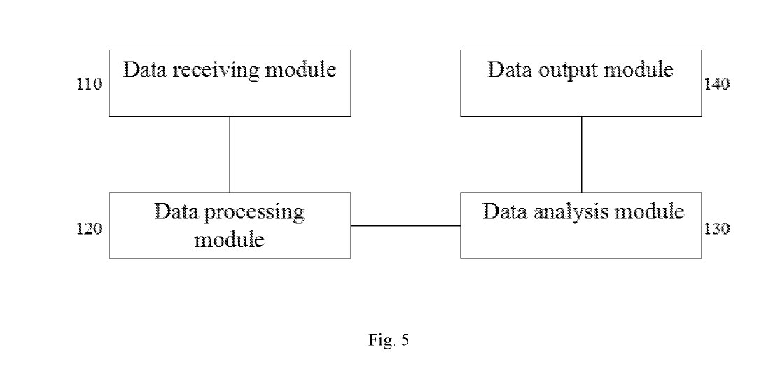

FIG. 5 is a schematic diagram of an underwear-based body data monitoring device in an embodiment. In this embodiment, the device may comprise a data receiving module 110, a data processing module 120, a data analysis module 130 and a data output module 140. The data receiving module 110 is configured to receive position data collected by a multi-axis sensor disposed on underwear and respiration data collected by a respiration sensor disposed on underwear. The data processing module 120 is configured to calculate displacement, velocity and acceleration according to the position data. The data analysis module 130 is configured to determine whether the displacement, velocity and acceleration all exceed a preset threshold, and further determine whether the respiration data is within a preset respiration range when the displacement, velocity and acceleration all exceed a preset threshold. The data output module 140 is configured to output alarm information when the respiration data is not within a preset respiration range.

In one embodiment, the data receiving module 110 is further configured to receive heartbeat data collected by a pulse sensor disposed on underwear and blood oxygen data collected by a blood oxygen sensor disposed on underwear. The data analysis module 130 is further configured to further determine whether the heartbeat data is within a preset heartbeat range and further determine whether the blood oxygen data is within a preset blood oxygen range, when the displacement, velocity and acceleration all exceed a preset threshold. The data output module 140 is further configured to output alarm information when the heartbeat range is not within a preset heartbeat range or when the blood oxygen data is not within a preset blood oxygen range.

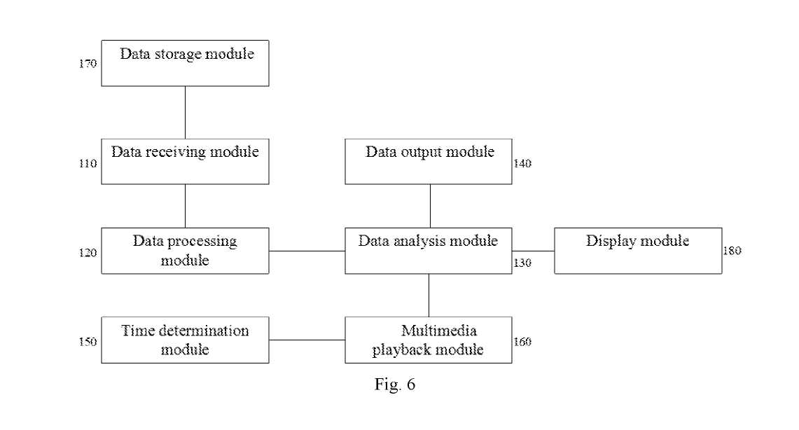

FIG. 6 is a schematic diagram of an underwear-based body data monitoring device in another embodiment. In this embodiment, the data receiving module 110 is further configured to receive tension data collected by a tension sensor disposed on underwear. The data analysis module 130 is further configured to determine whether the tension data is higher than a preset tension value, and further determine whether it is able to continuously receive body data collected by other sensors disposed on underwear when the tension data is higher than a preset tension value. The data output module 140 is further configured to output alarm information when it is unable to continuously receive body data collected by other sensors disposed on underwear.

In one embodiment, the device may further comprise a time determination module 150, a multimedia playback module 160 and a data storage module 170. The time determination module 150 is configured to determine whether a preset bedtime arrives or whether a preset wakeup time arrives. The multimedia playback module 160 is configured to play bedtime multimedia information when a preset bedtime arrives and stop playing the bedtime multimedia information when body data collected by sensors disposed on underwear is within a corresponding sleep data range, and play wakeup multimedia information when a preset wakeup time arrives. The data storage module 170 is configured to store body data collected by sensors disposed on underwear. The data analysis module 130 is further configured to determine whether body data collected by sensors disposed on underwear is within a corresponding sleep data range.

In one embodiment, the data receiving module 110 is further configured to receive temperature data collected by a temperature sensor disposed on underwear. The device may further comprise a display module 180. The display module 180 is configured to plot and display a 3D Cartesian coordinate system with temperature data as an x-axis, heartbeat data as a z-axis and respiration data as a y-axis, or plot and display a 3D Cartesian coordinate system with temperature data as an x-axis, heartbeat data as a z-axis and blood oxygen data as a y-axis.

In one embodiment, the data receiving module 110 is further configured to receive heartbeat sound data and ambient sound data collected by a sound recording device disposed on underwear. The heartbeat sound data and ambient sound data may be shown to the doctor, or sent to a family member or to the police together with the alarm information, so that the doctor, the family member or the police can understand the situation. The device will not be described repeatedly here. For the specific description of the device, please refer to the foregoing description.

The technical features of the embodiments mentioned above may be combined arbitrarily. For the sake of simplicity, not all possible combinations of the technical features of the embodiments mentioned above have been described. However, such combinations of the technical features shall be regarded as falling into the scope of this specification as long as they are not conflicted.

The embodiments mentioned above merely show several implementations of the present invention. Although these embodiments have been described specifically and in detail, these embodiments shall not be regarded as any limitation to the patent scope of the present invention. It should be noted that, a person of ordinary skill in the art may make various variations and improvements without departing from the concept of the present invention, and those variations and improvements shall fall into the protection scope of the present invention. Therefore, the protection scope of the present invention is subject to the appended claims.

What is claimed is:

1. An underwear-based body data monitoring method, comprising the following steps of:
receiving position data collected by a multi-axis sensor disposed on underwear and respiration data collected by a respiration sensor disposed on underwear;
calculating displacement, velocity and acceleration according to the position data;
determining whether the displacement, velocity and acceleration all exceed a preset threshold;
further determining whether the respiration data is within a preset respiration range when the displacement, velocity and acceleration all exceed a preset threshold; and
outputting alarm information when the respiration data is not within a preset respiration range.

2. The method according to claim 1, further comprising the following steps of:
receiving heartbeat data collected by a pulse sensor disposed on underwear and blood oxygen data collected by a blood oxygen sensor disposed on underwear; and
further determining whether the heartbeat data is within a preset heartbeat range and further determining whether the blood oxygen data is within a preset blood oxygen range, while performing the step of further determining whether the respiration data is within a preset respiration range; and
the step of outputting alarm information further comprises a step of:
outputting alarm information when the heartbeat range is not within a preset heartbeat range or when the blood oxygen data is not within a preset blood oxygen range.

3. The method according to claim 1, further comprising the following steps of:
receiving tension data collected by a tension sensor disposed on underwear;
determining whether the tension data is higher than a preset tension value;
further determining whether it is able to continuously receive body data collected by other sensors disposed on underwear, when the tension data is higher than a preset tension value; and
outputting alarm information when it is unable to continuously receive body data collected by other sensors disposed on underwear.

4. The method according to claim 1, further comprising the following steps of:
playing bedtime multimedia information when a preset bedtime arrives, and continuously receiving and storing body data collected by sensors disposed on underwear;
determining whether body data collected by sensors disposed on underwear is within a corresponding sleep data range;
stopping playing the bedtime multimedia information when body data collected by sensors disposed on underwear is within a corresponding sleep data range; and
playing wakeup multimedia information when a preset wakeup time arrives.

5. The method according to claim 2, further comprising the following steps of:
receiving temperature data collected by a temperature sensor disposed on underwear; and
plotting and displaying a 3D Cartesian coordinate system with temperature data as an x-axis, heartbeat data as a z-axis and respiration data as a y-axis, or plotting and displaying a 3D Cartesian coordinate system with temperature data as an x-axis, heartbeat data as a z-axis and blood oxygen data as a y-axis.

6. The method according to claim 2, further comprising the following steps of:
receiving heartbeat sound data and ambient sound data collected by a sound recording device disposed on underwear.

7. An underwear-based body data monitoring device, comprising:
a data receiving module configured to receive position data collected by a multi-axis sensor disposed on underwear and respiration data collected by a respiration sensor disposed on underwear;
a data processing module configured to calculate displacement, velocity and acceleration according to the position data;
a data analysis module configured to determine whether the displacement, velocity and acceleration all exceed a preset threshold, and further determine whether the respiration data is within a preset respiration range when the displacement, velocity and acceleration all exceed a preset threshold; and
a data output module configured to output alarm information when the respiration data is not within a preset respiration range.

8. The device according to claim 7, wherein the data receiving module is further configured to receive heartbeat data collected by a pulse sensor disposed on underwear and blood oxygen data collected by a blood oxygen sensor disposed on underwear;
the data analysis module is further configured to further determine whether the heartbeat data is within a preset heartbeat range and further determine whether the blood oxygen data is within a preset blood oxygen range, when the displacement, velocity and acceleration all exceed a preset threshold; and
the data output module is further configured to output alarm information when the heartbeat range is not within a preset heartbeat range or when the blood oxygen data is not within a preset blood oxygen range.

9. The device according to claim 7, wherein the data receiving module is further configured to receive tension data collected by a tension sensor disposed on underwear;
the data analysis module is further configured to determine whether the tension data is higher than a preset tension value, and further determine whether it is able to continuously receive body data collected by other sensors disposed on underwear when the tension data is higher than a preset tension value; and
the data output module is further configured to output alarm information when it is unable to continuously receive body data collected by other sensors disposed on underwear.

10. The device according to claim 7, further comprising:
a time determination module configured to determine whether a preset bedtime arrives or whether a preset wakeup time arrives;
a multimedia playback module configured to play bedtime multimedia information when a preset bedtime arrives and stop playing the bedtime multimedia information when body data collected by sensors disposed on underwear is within a corresponding sleep data range, and play wakeup multimedia information when a preset wakeup time arrives; and
a data storage module configured to store body data collected by sensors disposed on underwear; and
the data analysis module is further configured to determine whether body data collected by sensors disposed on underwear is within a corresponding sleep data range.

11. The device according to claim 8, wherein the data receiving module is further configured to receive temperature data collected by a temperature sensor disposed on underwear; and
the device further comprises:
a display module configured to plot and display a 3D Cartesian coordinate system with temperature data as an x-axis, heartbeat data as a z-axis and respiration data as a y-axis, or plot and display a 3D Cartesian coordinate system with temperature data as an x-axis, heartbeat data as a z-axis and blood oxygen data as a y-axis.

12. The device according to claim 8, wherein the data receiving module is further configured to receive heartbeat sound data and ambient sound data collected by a sound recording device disposed on underwear.
